(19) United States
(12) Patent Application Publication
(10) Pub. No.: US 2006/0154252 A1
(43) Pub. Date: Jul. 13, 2006

(54) Methods and composition for identifying therapeutic agents of atherosclerotic plaque lesions

(76) Inventors: Gerard Marguerie, Vitry-Sur-Seine (FR); Nora Benhabiles, Paris (FR)

Correspondence Address: YOUNG & THOMPSON, 745 SOUTH 23RD STREET, 2ND FLOOR, ARLINGTON, VA, 22202, US

(21) Appl. No.: 10/539,832

(22) PCT Filed: Dec. 19, 2003
(86) PCT No.: PCT/IB03/06419

§ 371 Date: Feb. 15, 2006

(30) Foreign Application Priority Data
Dec. 20, 2002 (EP) 022931992

(51) Int. Cl. C12Q 1/68

(52) U.S. Cl. 435/6

(57) ABSTRACT

The present invention relates to a method for identifying therapeutic agents for reducing and monitoring the growth, erosion, rupture or stability of an atherosclerotic plaque comprising the analysis of the differential expression of at least two genes coding proteins chosen among among Stearoyl CoA desaturase, Phosphatidic acid phosphate, and Phosphoinositide-specific-phospholipase-B1, eventually in association with the analysis of the differential expression of at least one gene coding a protein choosen in the group comprising Aldose reductase and aldehyde reductase, Sphingomyelinase, Acid ceramidase, Ceramide glucosyl transferase, Sphingosin phosphate liase, Thymosine beta 4, Aldehyde dehydogenase, ATPase Ca++ binding protein and CD163.

### FIELD OF THE INVENTION

The invention relates to cellular biology and pharmacology. The invention relates, generally, to the field of methods and composition for identifying compounds for reducing the accumulation of lipid rich vesicles in foam cells. The invention also relates to methods and composition for identifying therapeutic agents useful in human diseases in which accumulation of lipid laden cells is a pathogenic event. This includes atherosclerosis, hepatic steatosis, and obesity. The present invention more specifically describes novel methods of selecting or identifying new compounds that can modulate or reduce the growth, erosion and the rupture of arterial plaques. The invention also pertains to methods and compositions for monitoring the growth, erosion, rupture or stability of an atherosclerotic plaque as well as to methods and compositions for identifying therapeutic agents useful in humans for the treatment of atherosclerotic lesions in relation with the growth, erosion and rupture of an arterial plaque.

The present invention is based on the observation that a plurality of genes, more particularly three genes, that were not correlated before and not associated together during the progression of atherosclerosis, are differentially expressed during the progression of an atherosclerotic plaque relative to their normal expression and are co expressed with a new set of genes not known to be directly associated with atherosclerosis, and a series of referenced genes that have been associated with atherosclerosis. The three genes encode stearoyl coA desaturase (SCD), phosphatidic acid phosphates (PAP, EC 3.1.3.4) and Phosphoinositide-specific-phospholipase-B1 (PI-PLC, EC 3.1.4.11). These three enzyme are involved in the production and accumulation of diacylglycerides particles to form intracellular lipid vesicles. Together, they identify a new therapeutic pathway and exhibit target and/or marker gene characteristics for new methods for identifying compounds that can reduce the formation of lipid vesicles and for controlling plaque development at vascular sites that are prone to atherosclerosis.

### BACKGROUND OF THE INVENTION

Atherosclerosis is the most important cause of cardiovascular diseases and deaths in the industrialized countries (Ross. R. 1993, Nature, 362, 801-809). Coronary atherosclerosis is responsible for over 500 000 deaths annually in the United States and for a vast number of other clinical complications.

Atherosclerosis is the result of a complex unbalanced cellular and molecular reaction which normally functions as a defense mechanism in response to vascular injury. In pathological situations, however, this mechanism leads to endothelium dysfunction, cellular changes in the arterial intima and the continuous formation and growth of an arterial plaque containing lipids and foam cells.

Mechanisms controlling plaque growth and erosion and plaque rupture leading to thrombosis are unknown, and there is an unmet need for drugs in this area. The process appears to be the result of conflicting mechanisms. This includes for instance, lipid deposition and removal, cellular survival and death, cellular adhesion and extracellular matrix degradation and motility.

Atherosclerosis is initiated at specific sites by endothelium injury and dysfunction. Production of oxidized lipoproteins (oxLDL) and other oxidative and cytotoxic agents is probably the initial event that causes vascular injury. These agents have been shown to stimulate both survival and pro apoptotic mechanisms in endothelial cells and macrophages. These initial reactions occur during hyperlipidemia, dyslipidemia, hypertension, diabetes and fluctuating shear stress.

Endothelial dysfunction creates a chronic inflammation which results in a continuous recruitment of monocytes and macrophages. While beneficial in normal circumstances, this phenomenon may become pathologic and contribute to arterial plaque destabilization. The process is a slow reaction when compared to monocyte recruitment during infection, and may last a life time period. Activated endothelial cells and monocytes express scavenger receptors such as CD36 or LOX1 that bind and uptake modified LDL. This reaction leads to the formation of foam cells, destabilization of the arterial plaque and causes plaque rupture resulting in acute thrombosis.

The existence of endothelium dysfunction and the perpetuation of lipid deposition and foam cells accumulation are the most important consequences of vascular lesions in patients at risk. Particularly, the abundance of oxLDL is an important factor of atherosclerosis.

This can be controlled by modulating the activity of enzymes that are involved in cholesterol synthesis to reduce the accumulation of LDL and the toxic effect of oxLDL. Inhibitors that control the HMG-CoA reductase, exemplify and support this concept. These inhibitors have successfully been used for treating atherosclerosis. Only 35% of the patients, however, were shown to be responsive, and potential side effects were observed suggesting that individual dosage is probably a critical parameter.

Many of the drugs that are directed against these enzymatic pathways intend to treat the causes of atherosclerosis. But there is a need for drugs that can treat the consequences of atherosclerosis by controlling plaque growth and stability.

The development of an arterial plaque is complex and requires the-expression of many genes with multiple functions. The genes may be directly or indirectly involved in the process and may also be expressed in tissues other than vascular cells. To exhibit target characteristics, the gene must be directly involved in the pathogenesis of atherosclerosis.

### SUMMARY OF THE INVENTION

The invention is based on the discovery that Stearoyl CoA desaturase, Phosphatidic acid phosphatase and Phosphoinositide specific phospholipaseC-B1, are three enzymes implicated in the production and accumulation of diacylglycerides particles to form intracellular lipid vesicles are co-expressed and co-regulated, during the progression and the growth of a coronary atherosclerotic plaque. These three proteins are differentially expressed with a set of canonical genes encoding Aldose reductase, Aldehyde reductase, sphingomyelinase, acid ceramidase, Ceramide glucosyl transferase, sphingosin phosphate liase, thymosine beta 4, aldehyde dehydrogenase, ATP ase Ca++ binding protein and CD163. This set of canonical genes is up regulated in vivo in early atherosclerotic plaques both at the RNA and protein levels, and is co-expressed with reference genes that are known to be directly involved in the process of human atherosclerosis.

The present invention provides new methods and compositions for identifying molecules that can reduce the accumulation of lipid vesicles in a foam cell. These molecules can be used for reducing or monitoring the growth, erosion, rupture or stability of an atherosclerotic plaque.

The methods involve the analysis of the expression of at least two of three genes encoding Stearoyl CoA desaturase, phosphatidic acid phosphatase and Phosphoinositide specific phospholipaseC-B1, for monitoring the formation of lipid-vesicles-laden cells. The methods are based on the analysis of the differential expression of at least two of these genes. According to a particular embodiment, these three genes may be studied in association with at least one of the genes chosen among the canonical genes encoding Aldose reductase and Aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.

The present invention provides methods of screening or identifying compounds that modulate the formation of intracellular lipid vesicles comprising:

(1) contacting cells expressing at least two genes among Stearoyl CoA desaturase, phosphatidic acid phosphatase and Phosphoinositide specific phospholipaseC-B1 with one or several candidate compounds,

(2) measuring the formation of intracellular lipid vesicles in said cells, and

(3) comparing the amount of lipid vesicle formed in the presence of at least one substrate of one of the enzyme selected among Stearoyl CoA desaturase, phosphatidic acid phosphatase and Phosphoinositide specific phospholipaseC-B1 to select or identify compounds that reduce the formation, the size or the stability of the intracellular vesicles and the development of foam cells.

The present invention also provides a diagnostic method of atherosclerosis or cardiovascular disorders relating to the progression of an atherosclerotic plaque in a biological sample of a subject comprising the concomitant analysis of the differential expression of Stearoyl CoA desaturase, phosphatidic acid phosphatase and Phosphoinositide specific phospholipaseC-B1. This method may further comprises the analysis of the differential expression of at least one more gene coding a protein chosen among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.

The present invention relates to methods and compositions for identifying compounds useful for preventing or for reducing the accumulation of foam cells. These compounds can be used for the treatment of atherosclerotic lesions in relation with the growth, erosion and rupture of an arterial plaque. These compounds can also be used for the treatment of human diseases for which accumulation of lipid vesicles in specific cells represents a pathogenic event. This includes and is not limited to hepatic steatosis and obesity. The methods involve the detection of lipid vesicles formation and the concomitant analysis of Stearoyl CoA desaturase, phosphatidic acid phosphatase and Phosphoinositide specific phospholipaseC-B1, These three enzymes are implicated in the production and accumulation of intracellular diacylglyceride particles to form intracellular lipid vesicles. This set of genes may or may not be associated with the differential expression of at least one gene coding a protein chosen among Aldose reductase and Aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, in the presence of a test compound.

Such compounds are useful to monitor the progression or regression of the atherosclerotic plaque and to inhibit the accumulation of macrophages foam cells at sites of vascular lesions when large amount of LDL and oxLDL are present. Therefore, the invention relates to the use of a compound modulating the combined expression of at least two protein among Stearoyl CoA desaturase, phosphatidic acid phosphatase and Phosphoinositide specific phospholipaseC-B1, with or without at least one gene coding a protein chosen among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, or modulating the activity of said at least two proteins for the preparation of a pharmaceutical composition useful for preventing and/or treating artherosclerosis or cardiovascular disorders relating to the atherosclerotic plaque.

### BRIEF DESCRIPTION OF THE FIGURES

FIG. 1 shows cross-sections through the left anterior descending (LAD) coronary aorta root of pigs under control and 4% cholesterol rich diet conditions. Sections A to F are representative of advanced atherosclerotic plaque at 6, 9 and 12 weeks (A,C,E, ×10; B,D,F, ×40). Lipids were stained with oil red'O, (A and B), cells were labelled with toluidine blue (C,D,E,F)

FIG. 2 shows details of early fibro-fatty plaques in the left anterior descending coronary aorta root of pigs, and illustrates the accumulation of macrophages foam cells loaded with lipid vesicles

FIG. 3 shows the plasma lipoprotein profile of hyperlipidemic pigs fed with a 4% cholesterol diet for 6, 9 and 12 weeks. Data represent mean value for 10 pigs

FIG. 4 shows laser microdissected sections from early advanced plaques and RNA extraction from hypercholesterolemic pig. A: Section from LAD; B: Microdissected section; C: RNA extraction and analysis showing a high quality ratio between 18S and 28S fractions.

FIG. 5 shows the amplification of mRNA from laser micro-dissected sections of the plaque. Panel A, Antisens RNA were amplified by two round of in vitro transcription. The factor of amplification was around 80 000. Panel B, Medium size of RNA was about 1400 nucleotides. Panel C, Linearity of this amplification reaction was estimated, using RT-PCR amplification of low, medium and high activity genes

FIG. 6 shows a typical expression signature on a human DNA chip containing 12 000 different genes probes.

FIG. 7 shows a factorial analysis of 24 different pigs, including control pigs and diet pigs, with a set of 1200 genes that were found to be up or down regulated during the progression of the fibro-fatty plaque. This analysis clearly identifies three groups of animals. These groups can be clustered with phenotypic attributes which characterize the progression of the plaque and the content of lipid in the plaque.

FIG. 8 shows a prototypic permanent cell line with a foam cell phenotype, illustrating the accumulation of lipid vesicles. Macrophage permanent cells were cultured in the presence of labeled oxidized LDL.

FIG. 9 illustrates the induction of lipid vesicles in differentiated macrophage permanent cell line. The formation of vesicles is obtained by culturing the cell line in the presence of 200 μM of albumin-coupled stearic acid.

FIG. 10 shows inhibition of vesicles accumulation in a typical foam cell using a specific inhibitor to the stearoyl coA desaturase inhibitor of at least one of the protein.

### DETAILED DESCRIPTION

The present invention provides a set of three genes hereafter called “new genes” involved in the accumulation of lipid vesicles that are concomitantly up regulated in macrophage foam cells during the progression of a fibro-fatty arterial plaque. The present invention also providees a series of other genes, hereafter also called canonical genes, that is similarly differentially expressed relative to their normal expression in early and advanced atherosclerotic plaques containing macrophages foam cells, under hyperlypidemic conditions. These two series of genes identify new pathways and exhibit target and/or marker gene characteristics for controlling or reducing plaque development at vascular sites that are prone to atherosclerosis.

The present invention provides methods and compositions for controlling or reducing atherosclerotic plaque progression and erosion, and their clinical complications. The invention is based on the discovery that Stearoyl CoA desaturase, phosphatidic acid phosphates and Phosphoinositide-specific-phospholipase-B1are co-regulated during the accumulation of foam cells in an arterial fibro-fatty plaque. These three enzymes are implicated in the production and accumulation of diacylglycerides particles to form intracellular lipid vesicles. The invention is also based on the discovery that this cluster of three genes is co-regulated with a set of canonical genes encoding Aldose reductase and aldehyde reductase, sphingomyelinase, acid ceramidase, Ceramide glucosyl transferase, sphingosin phosphate liase, thymosine beta 4, aldehyde dehydrogenase, ATP ase Ca++ binding protein and CD163 and a series of reference genes that are known to be directly involved in the process of human atherosclerosis.

The present invention provides methods of screening or identifying compounds that modulate the formation of intracellular lipid vesicles comprising (1) separately contacting cells expressing at least two genes among Stearoyl CoA desaturase, phosphatidic acid phosphatase and Phosphoinositide specific phospholipaseC-B1 in association or not with at least one gene coding for a protein chosen among the canonical genes coding for Aldose reductase and Aldehyde reductase, Sphingomyelinase, Acid ceramidase, Ceramide glucosyl transferase, Sphingosin: phosphate liase, Thymosine beta 4, Aldehyde dehydrogenase, ATPase Ca++ binding protein and CD163, (2) measuring the formation of vesicles in the presence of one or several candidate compounds, and (3), comparing the amount of lipid vesicle formed in the presence of one or several compounds and at least one substrate of one of the enzyme selected among Stearoyl CoA desaturase, phosphatidic acid phosphatase and Phosphoinositide specific phospholipaseC-B1 to select or identify compounds that reduce the formation, the size or the stability of the intracellular vesicles.

The present invention relates to methods and compositions to monitor the progression or the regression of plaques and to inhibit the accumulation of macrophages foam cells at sites of vascular lesions when large amount of LDL and ox LDL are present. The method comprises the analysis of the differential expression of at least two of the new genes coding a protein chosen among Stearoyl CoA desaturase, phosphatidic acid phosphates and Phosphoinositide-specific-phospholipase-B1 in association or not with at least one canonical gene coding a protein among Aldose reductase and Aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.

Said analysis is carried out in human or animal cells, tissue sections or animal models.

Discussed below are methods for prognostic and diagnostic evaluation of atherosclerosis, including the identification of subjects exhibiting a predisposition to atherosclerosis and the imaging of an atherosclerotic plaque. The invention provides a diagnostic method of artherosclerosis or cardiovascular disorders relating to the atherosclerotic plaque in a biological sample of a subject comprising the analysis of the differential expression of at least two gene coding a protein chosen among Stearoyl CoA desaturase, phosphatidic acid phosphates and Phosphoinositide-specific-phospholipase-B1, in association or not with at least one gene coding for one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.

Said analysis is carried out in human or animal cells or tissue sections.

According to another embodiment, the method of the invention comprises:

providing a plurality of different ligands in the form of an array on a solid surface, said different ligands being complementary to different segments of at least two genes coding a protein chosen among Stearoyl CoA desaturase, phosphatidic acid phosphates and Phosphoinositide-specific-phospholipase-B1, in association or not with at least one gene coding for one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 or being complementary to different segments of at least one gene coding said proteins,

applying a sample solution potentially containing the targets of the ligands to the array of ligands under conditions which allow the interaction of said ligands and its target, and

measuring the interactions of the targets with the different ligands of the array.

In preferred embodiments, the ligands are nucleic acid probes and the sample contains target nucleic acids in order to measure the hybridization of the probes with the target nucleic acids. Advantageously, the nucleic acid probes are oligonucleotides.

Additional embodiments of the invention provides array comprising 2 to about 200 oligonucleotides localized in discrete location per square centimeter on the solid surface.

The sample is for example from a patient developing artherosclerotic plaque.

The methods of the invention comprises the measure of the differential expression of at least two genes coding a protein chosen among Stearoyl CoA desaturase, phosphatidic acid phosphates and Phosphoinositide-specific-phospholipase-B1, in association or not with at least one gene coding for one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 and the comparison of said measure with the normal expression of said protein in early and advanced atherosclerotic plaques containing macrophages, under hyperlypidemic conditions and in the absence of high levels of blood glucose and insulin.

The new set of three genes and the canonical genes are co-expressed with reference genes known to be differentially expressed during the progression of atherosclerotic plaques in mammals and humans. According to the present invention, these reference genes are utilized in combination with the set of canonical genes and the set of three genes to profile the degree of progression of the plaque.

Reference genes refer to a set of genes that have already been described to be expressed in human atherosclerotic plaque. Canonical genes refer to genes coding for Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163. The new set of genes refer to Stearoyl CoA deasturase, phosphatidic acid phosphates and Phosphoinositide-specific-phospholipase-B1, which have not yet been described as being involved in the progression of a fibro-fatty plaque. The association of this new set of genes with the canonical genes and the reference genes, define a typical signature for an atherosclerotic target molecule. For each of the known target genes, an average of the fold changes was evaluated. Novel genes, and canonical genes are associated with the development of an atherosclerotic plaque and were characterized in reference to this set of reference genes and exhibited expression patterns similar to these reference genes and were significantly and statistically differentially induced when compared with genes from non atherosclerotic vascular endothelium located at the same position in the coronary artery or non stimulated circulating monocytes.

The set of reference genes that are representative of an atherosclerotic plaque includes but is not limited to : membrane associated genes such as CD68, CD36 which are both markers of the macrophage lineage; PECAM 1, a marker for endothelial cells; markers of the inflammatory response such as TLR4, HSP60 and HSP70, Galectin 3 and IL1-R; markers of the oxidative stress including HIF-1 and Paraoxanase 3, metabolic marker such as NADH dehydrogenase; lipoprotein receptors such as LDL-R and VLDL-R.

Also discussed below are methods for detecting agents that may control the activity of these proteins in relation to the accumulation of lipid vesicles in foam cells at sites of an atherosclerotic lesion.

Therefore, the invention relates to method of screening compounds useful for the treatment of artherosclerosis or cardiovascular disorders relating to the atherosclerotic plaque comprising the quantification of lipid vesicles in a foam cells in association with the analysis of the differential expression of at least two gene coding a protein chosen among Stearoyl CoA desaturase, Phosphatidic acid phosphate, and Phosphoinositide-specific-phospholipase-B1, in association or not with at least one gene coding for one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163. gene coding a protein chosen among Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, in the presence of a test compound.

Said analysis is carried out in human or animal cells, tissue sections or animal models. It can be also performed on a solid support for high throughput methods. In such embodiments, the invention comprises:

providing a plurality of different ligands in the form of an array on a solid surface, said different ligands consisting of all or part of at least two proteins chosen Stearoyl CoA desaturase, Phosphatidic acid phosphate, and Phosphoinositide-specific-phospholipase-B1, in association or not with at least one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163,

applying a solution containing a test compound to the array of ligands, and

measuring the interaction, such as the binding, of the test compound with the different ligands of the array.

The test compounds may be proteins or molecule of small molecular weight.

The analysis according to the above methods of the present invention may be performed at the mRNA or protein level.

A method of screening compounds useful for the treatment of artherosclerosis or cardiovascular disorders relating to the atherosclerotic plaque, according to the present invention comprises:

providing an assay for at least two proteins chosen among Stearoyl CoA desaturase, Phosphatidic acid phosphate, and Phosphoinositide-specific-phospholipase-B1, in association or not with at least one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, in the presence of a test compound.

contacting said assay with a test compound, and

measuring the action of the test compound on the said protein in the assay.

The new three genes identify also new ways to treat patients with hypercholesterolemia induced atherosclerotic plaques. Therefore the invention relates to the use of a compound modulating the expression of at least two genes coding a protein chosen among Stearoyl CoA desaturase, Phosphatidic acid phosphate, and Phosphoinositide-specific-phospholipase-B1, in association or not with at least one gene coding for one protein among Aldose reductase and Aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, or modulating the activity of said at least two protein for the preparation of a pharmaceutical composition useful for preventing and/or treating artherosclerosis or cardiovascular disorders relating to the atherosclerotic plaque.

1) Identification of at Least Two of the Proteins in an Atherosclerotic Plaque and Differential Expression During the Progression of the Plaque

Differential expression refers to both, quantitative and qualitative differences in at least two of the proteins, mRNA and protein expression using vascular tissues containing atherosclerotic lesions or circulating cells in pro-atherogenic situations such as hypercholesterolemia. The gene may be activated or down regulated in normal vessel wall versus atherosclerotic plaque or in atherogenic circulating cells versus normal cells. The later may include for instance circulating monocyte in atherogenic conditions versus normal monocytes. Differential expression may be detected via differential techniques, including RT-PCR, northern analysis, DNA micro-arrays and DNA chips, differential expression libraries, immuno-histochemistry, two dimension electrophoresis, and mass spectroscopy. Differential expression also refers to expression that can be used as part of prognostic or diagnostic tools that may be useful to monitor the development of an arterial plaque in atherosclerosis.

At least two of the proteins can be used as target gene. This refers to a differential expression involved in atherosclerosis in a manner that can modulate the level of gene expression or activity to modulate and ameliorate the stability of an arterial plaque. This method can be applied in different experimental paradigms such as those described below:

Foam Cells: Gene differential expression or protein activity of at least two of the proteins may be used to quantitatively or qualitatively detect genes as secondary targets that are co-regulated during the maturation of macrophages and the formation of foam cells under circumstances that mimic the development of an atherosclerotic plaque. This may include for instance, but is not limited to, the presence of Lipoproteins and modified lipoproteins or components from hyperlipidemic serum. Differential expression of at least one of the protein may be used to validate an ex vivo model. The definition of foam cells can be extended, but is not limited to, to cells that can accumulate lipid vesicles, such as hepatocytes, adipocytes and smooth muscle cells.

Endothelial dysfunction: Endothelial cell monolayer can be used to monitor gene expression or protein activity that may be correlated with a differential expression and activity of at least two of the proteins and may have target characteristics under circumstances that mimic atherosclerosis. At sites of atherosclerosis, for instance, endothelial cells activate and stimulate the expression of survival effectors as well as pro-apoptotic agents. Endothelial cells also activate the expression of adhesive molecules. Differential expression of at least two the proteins may be used to monitor the expression of these genes and to validate ex vivo atherosclerotic phenotypes in cell based screening models under conditions that stimulate vascular injury. This may include HUVEC and BAEC as well as permanent cell line exhibiting endothelial cell phenotype. Cultured monolayers can also be exposed to fluctuating shear stress in specialized apparatus.

Detection of mRNA: To detect differentially expressed proteins and associated genes, mRNA can be isolated and amplified from tissue section, cell extract or biopsies, using routine protocols in the art. Transcript within the RNA sample may be detected by utilizing hybridization technologies such as DNA chip technology containing specific probe sequences or RT PCR using specific oligonucleotides that are specifically designed to monitor the differential expression of the gene. Expression can then be corroborated with routine technologies including quantitative RT-PCR or northern blot analysis.

Detection of protein: The presence of at least two of the proteins can be detected in atherosclerotic tissues by routine immuno-histochemistry. The protein can also be detected via an ELISA assay or utilizing mass spectroscopic technologies following protein isolation in a two dimensional gel eclectrophoresis apparatus. The two hybrid system may also be used to detect intracellular proteins that may associate with at least one of the protein during the development of an arterial plaque and the formation of foam cells.

2) Inhibitors for Controlling Differential Expression of at Least Two of the Proteins During Atherosclerotic Plaque Growth and Erosion

Methods that can be used for the identification of agents controlling the expression and activity of at least one protein of the group in a growing arterial plaque are multiple.

Cell based assays: the proteins or their mRNA may be used to identify molecular entities that modulate the formation of a foam cells using macrophages or permanent cell lines based screening assays in conditions that induce lipid vesicle formation and reproduce the development of an atherosclerotic plaque. This may include but not limited to, THP1 cells (ATCC# TIB-202, U937 cells (ATTCC# CRL1593). Monocyte/macrophages but also hepatocytes, adipocytes and smooth muscle cells may be isolated using routine protocols and stimulated with but not limited to, oxLDL or any modified lipoprotein, and components from hyperlipidemic serum. Either one of the molecules, may also be used in a screening assay for the identification of agents that can protect against endothelium dysfunction. Sources of endothelial cells may be, but not limited to, HUVEC or BAEC.

These cell based assays may be phenotyped as atherosclerotic cells, using differential expression of at least two proteins or mRNA expression in association with the accumulation of vesicles and the expression of atherosclerosis associated genes and used to detect novel associated genes.

These cell based assays may also be used to screen for compounds that are capable of controlling the expression of at least two genes coding two proteins chosen among Stearoyl CoA desaturase, Phosphatidic acid phosphate, and Phosphoinositide-specific-phospholipase-B1, in association or not with at least one protein among, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163, and/or corresponding protein, and limiting the growth and instability of an atherosclerotic lesion. Thus, cell based assays using the detection of vesicles and the differential expression of the above genes may be used to identify drugs, pharmaceuticals, therapies, and interventions which may be effective in treating arterial plaque growth and rupture as well as steatosis and obesity.

Animal based systems: Animal based systems may include genetically modified or not modified animals. Recombinant animal models may include, but is not limited to, LDL-Receptor, ApoE and ApoB deficient mice, ApoR deficient pigs. Non recombinant animal model may include rabbit, rat, mouse and pigs. The expression of at least two of the proteins in these animal models may be used for phenotyping, and strain selection for atherosclerotic steatosis and obesity diseases.

The example presented hereafter demonstrates the generation, phenotypic characterization and usefulness of pig expressing of at least two of the following proteins in an early atherosclerotic lesion. Differential expression in these animals may be used for screening, validation and optimization of drug candidates.

3) Assays for Compounds That Interfere with Interaction of at Least One of the Protein and Other Cellular Compounds

Proteins that are differentially expressed, may in vivo, interact with one or more intracellular compounds within an atherosclerotic tissue. Those compounds may include intracellular proteins, phospholipids, fatty acids, and small molecules. Agents that can interfere with these interactions may be useful in regulating vesicle formation,foam cell formation and plaque growth and stability. Any assay system which will allow interaction of at least one of the protein and cellular compounds under circumstances that mimic the development of an atherosclerotic lesion or from an atherosclerotic plaque versus vascular cells from non atherosclerotic vessel wall, will be convenient. Alternatively, arrays containing at least two of the proteins chosen among the proteins cited above, in combination, may be used to screen for molecules that can interact with at least one of said protein. Therefore, protein arrays will be convenient. The formation and the inhibition of the complex can be quantitatively or qualitatively detected using fluorescent labeling. The reaction can be conducted in a solid phase assay or in a liquid phase. Antibodies can be used as a signal amplifier either in the liquid phase or in the solid phase.

4) Monitoring of Effects During Clinical Trials

Monitoring the effect of a drug candidate for treating atherosclerotic growth and plaque instability using tools to detect differential expression at least two of the proteins may be applied in clinical trials. For example, differential expression may be used to study drug efficacy in human tissue section by immunohistochemistry or in situ hybidization. Expression may be or not associated with plaque imaging and be used for monitoring patients at risk.

5) Antibodies with Potential Therapeutic Activity in Atherosclerosis

Antibodies that modulate differential expression of at least two of the proteins in arterial lesions and can interfere with the cellular activity of these proteins in an atherosclerotic plaque, may be used for controlling plaque growth and stability. Such antibodies include polyclonal antibodies, murine and human monoclonal antibodies, single chain antibodies, Fab fragments and chimeric antibodies.

6) Imaging Atherosclerotic Plaque

As shown in the present invention, at least two of the new genes are up regulated in the vascular wall at sites that are prone to develop an atherosclerotic lesion. Differential expression of these proteins may thus be used for non invasive imaging of the growth, erosion and stability of an arterial plaque at sites of ischemia. As described in the example hereafter, Stearoyl CoA desaturase, Phosphatidic acid phosphate, and Phosphoinositide-specific-phospholipase-B1, in association or not with at least one protein among, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 are up regulated in a plaque and can be used to label endothelial cells or foam cells within the plaque. This may constitute an excellent tool for monitoring the development and/or the regression of the plaque and to develop an appropriate therapeutic strategy.

Non invasive imaging can be performed with different marker including monoclonal antibodies labeled with radioisotopes or specific ligand that can be designed based on the structural parameters of stearoyl CoA desaturase.

### EXAMPLES

The following examples are offered to illustrate the invention, but not to limit the present invention.

### Example 1

### Animal Model and Sample Preparation

Differential gene expression analysis during the progression of an atherosclerotic plaque may be applied to a variety of animal models for the detection of co regulated pathways that may constitute targets implicated in the growth and the erosion of atherosclerotic lesions. These animals may be used for screening or validation of molecules that can modulate the differential expression of at least two of the protein chosen among Stearoyl CoA desaturase, Phosphatidic acid phosphate, and Phosphoinositide-specific-phospholipase-B1, in association or not with at least one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 at the level of an arterial plaque. Animal based systems may include non genetic and genetic modified animals such as, but not limited, pigs, mouse, rat, rabbit, ApoE negative mouse, ApoB negative mice and ApoR mutant pig.

In the present invention, a mini pig model was used to monitor the differential expression of genes during the development of an atherosclerotic plaque under dietary supplementation using a cholesterol rich diet.

These mini pigs were obtained by crossbreeding Gottinger and Yucatan minipigs (Charles-River laboratories). They were housed in a temperature-controlled room (to 20±1° C.) at 50±2% humidity on a 12-hour/12-hour light/dark cycle. The investigation was in conformity with the Guide for the Care and Use of Laboratory Animals published by the US National Institutes of Health (NIH Publication No. 85-23, revised 1996). All experimental procedures for these animals were performed in accordance with protocols approved by the Institutional Animal Care and Research Advisory Committee.

Atherosclerosis was induced by feeding the animals a diet containing 4% cholesterol, 14% beef tallow, and 1% hog bile extract in daily amounts of 1000 g. Water was provided ad libidum. The fatty acid composition of the beef tallow is summarized in table 1 hereunder.

TABLE 1
fatty acid composition of The beef tallow

| Fatty acid | % |
|---|---|
| C16:0 | 14 |
| C18:0 | 4.3 |
| C18:1 | 26 |
| C18:2 n-6 | 51 |

1) Cardiac Catheterization

Immediately prior to sacrifice the animals were sedated with 1 mL azaperone IM (Stresnil 40 mg/mL, Janssen Pharmaceutica) and premedicated with 7 mg/kg ketamine IM (Imalgene 100 mg/mL, Janssen). The animals were incubated and artificially ventilated with a mixture of 30% oxygen and 70% room air (Mark 7A Bird respirator). Arterial blood gases were checked at regular intervals and the ventilation adjusted to maintain normal blood gas values. Anesthesia was maintained by a continuous intravenous infusion of sodium pentobarbital (Nembutal 60 mg/mL, Signify) at a rate of 3 mg·kg⁻¹·h⁻¹. Arterial access was achieved by surgical isolation and cannulation of the left carotid artery. The animals were then given 200 IU/kg heparin and 1 mg/kg IV of 2% lidocaine (Xylocaine 20 mg/mL, Astra) before manipulation of the coronary arteries.

2) LAD Sections

Briefly, LAD was perfused with cold NaCl 0.9% via aortic root, carefully dissected and cut into 7-μm sections.

For gene expression studies, LAD was embedded in OCT and snap frozen in liquid nitrogen until sectioning. For histological and immuno-histological analysis, LAD was transferred to embedding cassette in methanol 70% till paraffin embedding

3 ) Histo-Morphometric and Immunohisto-Chemical Analysis

Seven μm sections of the proximal LAD were stained with hematoxylline-eosine to assess lesion size. Morphometric analysis of sections was performed using the Leica Quantimet 600 image analysis system (Leica, Brussels, Belgium). The external elastic lamina area (EEL), internal elastic lamina area (IEL), medial, intimal, and luminal areas are measured. Total lipid deposition in the lesions was determined using oil-red-O staining. The total amount of collagen in the lesion was determined on picrosirius red stained sections viewed in normal light. Triple helix collagen was measured on the same sections viewed in polarized light. Elastin content was measured on Verhoeffs-stained sections and by measuring auto-fluorescence of the coronary lesion. Atherosclerotic lesions were classified using the Stary classification into early lesions and more advanced lesions.

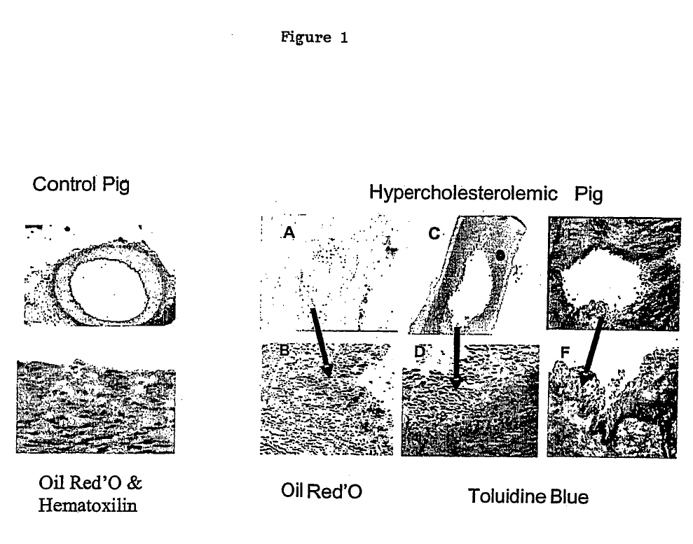

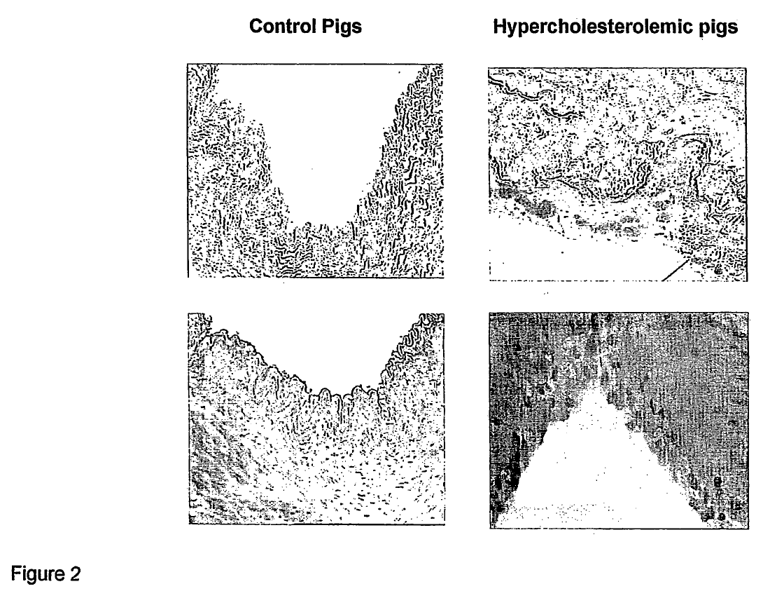

Examples of arterial cross sections showing early and advanced plaques containing lipids and macrophages are illustrated in FIG. 1 and FIG. 2.

4) Seric Lipids and Glucose Measurement

Peripheral venous blood was drawn from an ear vein. Total cholesterol, HDL cholesterol and triglyceride levels were measured by enzymatic methods (Boehringer Mannheim, France). LDL cholesterol levels were calculated with the Friedewald formula. Plasma oxidized LDL (ox-LDL) was measured with a mAb-4E6 based competition ELISA. The monoclonal antibody is directed against a conformational epitope in the apoB-100 moiety of LDL that is generated as a consequence of substitution of lysine residues of apoB-100 with aldehyde residues. The C50 values, i.e. concentrations that are required to obtain 50% inhibition of antibody binding in the ELISA, are 25 mg/dL for native LDL, and 0.025 mg/dL for oxidized LDL with at least 60 aldehyde-substituted lysines per apoB-100.

Plasma levels of C-Reactive-Protein (CRP) was measured with an immuno turbidimetric assay (Roche) with a detection limit of 3 mg/l.

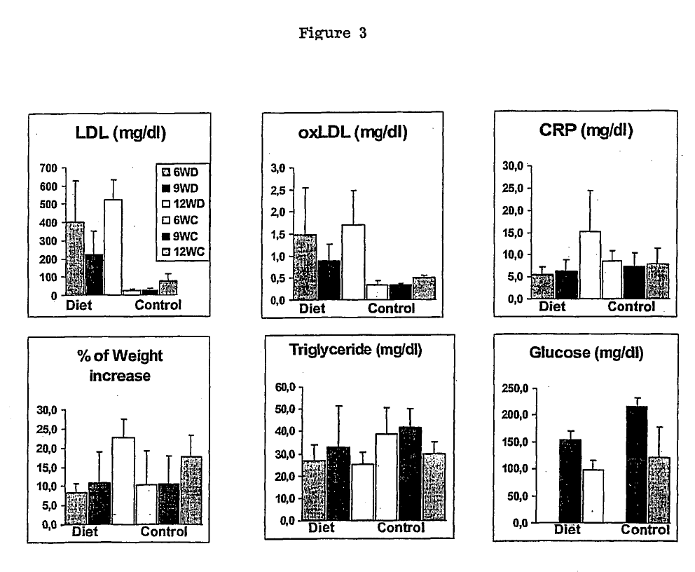

FIG. 3 illustrates the different parameters of this pig model, indicating that this animal model is a true hyper-cholesterolemic model with absence of hyperglycemia and hypertriglycerimia.

5) Monocytes Isolation

Blood was drawn into 4% sodium citrate and centrifuge 10 min, washed two times in HBSS at 3120 g (4500 rpm), 10 min at 20° C. Leukocyte isolation was performed by a ficoll -Histopaque Gradient (1.119) as described by the provider (Sigma,) and monocytes were isolated using CD14 magnetic microbeads (Miltenyi). Cells were washed twice, lysed with trizol reagent and stored at −80° C.

6) Plague Extraction and mRNA Amplification

To be physiologically relevant and to be associated with the progression of a plaque, differential gene expression must be quantitatively detected at the level of cells that are recruited during the growth of an atherosclerotic plaque. This can be monitored with microdissection technologies. In the present invention, the following method was used:

Laser capture micro-dissection (LCM): LAD were sectioned at 8 μm in a cryostat, mounted on polylysine coated glass slides (two sections per slides). The slides then were stored at −80° C.

For lesions phenotyping, one every 20 (twenty) slides was stained by Oil Red O (slides were dipped just before defrosting in ORO solution (72 mg ORO, 24 ml isopropanol, 16 ml RNase-free water) for 10 minutes and rinse in two bathes of H2O). The following slides were stained by Toluidine blue (slides were dipped just before defrosting in 75%EtOH for 4 sec, stained in a bath of Toluidine blue (dissolved at 0.1%w/v in PBS) solution for 8 sec, rinse in RNase-free water, deshydrated in 75% ethanol for 30 sec).

Before microdissection, the frozen sections were fixed in 75% ethanol for 30 sec, rinsed in RNase free water in order to remove the OCT, dehydrated for 30sec in 75%, 95% and 100% ethanol and 3 min in xylene successively. Once air-dried, the tissues were laser-capture microdissected by a PixCell II LCM system using Capsure HS LCM caps following the manufacturer's protocols (Arcturus Engineering, Mountain View, Calif.).

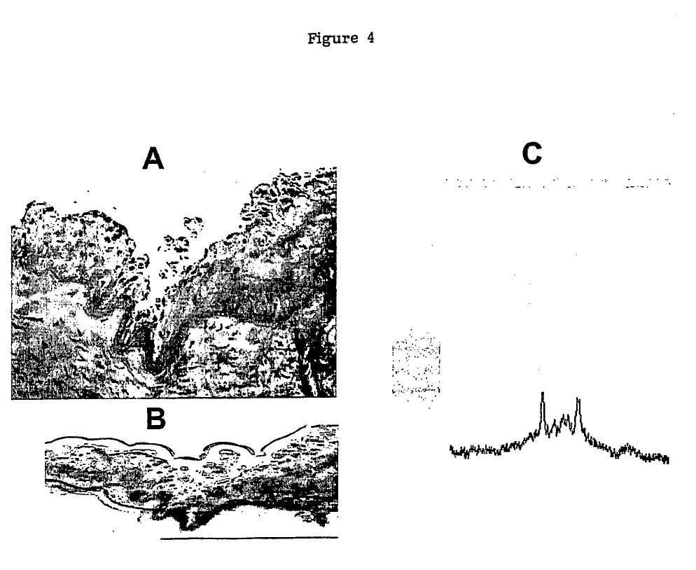

A typical plaque capture experiment is illustrated in FIG. 4.

RNA extraction: Total RNAs were extracted from either circulating monocytes or laser-capture cells from one entire LAD section with the RNeasy Mini Kit (Qiagen) according to the manufacturer's recommendations.

Total RNA from monocytes was extracted using Trizol solution (In vitrogen) and PLGI-Heavy Phase Lock gel (Eppendorf).

Optical density was measured for each sample with a biophotometer (Eppendorf) using disposable cuvettes.

Quality of the tRNA preparation from monocytes and from one entire section of LAD was visualized with the eukaryote total RNA nano assay using the Agilent 2100 Bianalyzer (following the manufacturer's protocols).

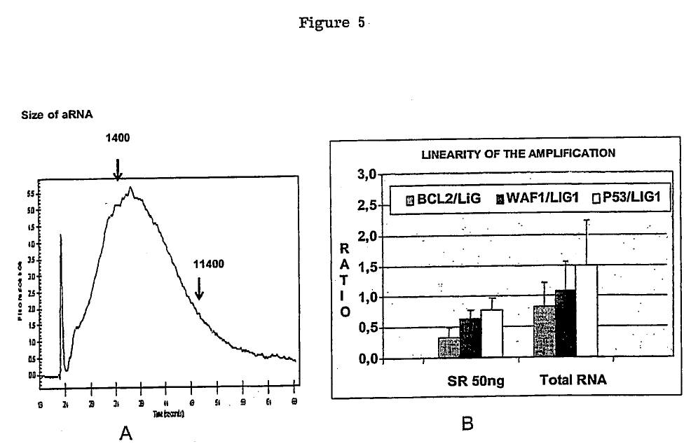

FIG. 5 exemplifies the quality of the capture and of the RNA extract.

cDNA synthesis: All purified RNA from microdissected cells or 500 ng to 5 μg of monocytes tRNA was mixed with 1 μl of 10 mM dNTP mix and 1 μl of 20 mM T7-(dT) 24 primer in 10 μl final volume, incubated for 5 minutes at 65° C. and chilled on ice. Next, 4 ul of 5× First-strand reaction Buffer, 2 μl of 0.1 M DTT and 1 μl RNaseOUT Recombinant Rnase Inhibitor (40 U/ul) were added and placed at 42° C. for two minutes, 200 U of Superscript II RNase H⁻ RT (In vitrogen) were added and the reaction kept at 42° C. for 1 hour. Next, 30 μl 5× second strand reaction buffer, 10 mM dNTP mix (3 μl), 4 μl DNA polymerase I (10 U/μl), 1 μl E. Coli DNA ligase (10 U/μl), 1 μl RNase H (2 U/μl) and 91 μl of RNase-free water were added and the reaction mixture was incubated at 16° C. for 2 h, followed by incubation of 10 min at 16° C. after addition of 2 μl of T4 DNA polymerase (5 U/μl). The reaction was stopped by adding 10 μl of EDTA 0.5M. Next, the cDNA was extracted with phenol-chloroform-isoamyl alcohol using PLGI-light Phase Lock gel, and precipitated with NH4OAc and ethanol in-presence of 5g of glycogen.

T7 RNA polymerase amplification (aRNA): The MEGAscript™ T7 kit (Ambion) was used: 8 μl double-stranded cDNA, 2 μl Ambion transcription buffer, 2 μl each of 150 mM ATP, CTP, GTP and UTP and 2 μl Ambion T7 Enzyme mix were mixed and incubated at 37° C. for 6 hours. Next, aRNA were extracted with phenol-chloroform-isoamyl alcohol using PLGI-Heavy Phase Lock gel and cleaned up using RNeasy Mini Kit. The volume was reduced in a speed vac.

Second round of aRNA amplification First, aRNA (from first round amplification was mixed with 250 ng random hexamer and 1 μl of 10 mM dNTP mix, incubated at 65° C. for 5 minutes and then chilled on ice. Next, 4 ul of 5× First-strand reaction Buffer, 2 μl of 0.1 M DTT and 1 μl RNaseOUT Recombinant Rnase Inhibitor (40 U/ul) were added. The reaction was left to equilibrate at room temperature before to add 200 U of Superscript II RNase H⁻ RT (In vitrogen), the reaction was then incubated first at room temperature for 10 min then at 42° C. for 50 min. Then, 1 μl of RNase H was added and the reaction incubated at 37° C. for 20 min, after which the reaction was heated to 95° C. for 2 min and chilled on ice. For second strand cDNA synthesis, 2 μl of 20 μM T7-(dT)24 primer were added and the mixture incubated at 70° C. for 5 min and 42° C. for 10 min. Next, 30 μl 5× second strand reaction buffer, 10 mM dNTP mix (3 μl), 4 μl E. Coli DNA polymerase I (10 U/μl), 1 μl RNase H (2 U/μl) and 89 μl of RNase-free water were added and the reaction mixture was incubated at 16° C. for 2 h. Then, 2 μl of T4 DNA polymerase (5 U/μl) were added and the reaction incubated at 16° C. for 10 more min before to be stopped by the addition of 10 μl of 0.5M EDTA. The double stranded cDNA was extracted with phenol-chloroform-isoamyl alcohol using PLGI-light Phase Lock gel to get rid of proteins, and precipitated with NH4OAc and ethanol in presence of 5 μg of glycogen. The cDNA was then resuspended in 8 μl RNAse-free water and use for second-round T7 in vitro transcription as above except that the incubation last only three hours at 37° C.

After phenol-chloroform-isoamyl alcohol and RNeasy Mini Kit cleanup of the aRNA, the density optic was measured and the concentration and the size distribution of aRNA was analysed with the mRNA smear nano assay using the Agilent 2100 bioanalyzer (following the manufacturer's protocols).

Following a second run of amplification the aRNA sample was tested for quality. This included, size distribution and preservation of the relative abundance of the RNA. FIG. 5 illustrates these quality controls. The relative abundance of aRNA was certified using low, medium and high activity gene markers.

### Example 2

### Differential Expression

Differential expression of genes in a given sample can be monitored with different technologies including, traditional northern blot, RT-PCR, and differential display. However, methods and assays of the invention are most efficiently designed with array and DNA-chip technologies.

Any hybridization format may be used, including solution based and solid support based formats. In the present example, a high density array of DNA probes on a solid support was preferred with the following protocol.

1) Preparation of Pig Universal Reference

A pig universal reference was made. Total RNA was extracted with Qiagen RNeasy (Qiagen) from 8 swine control organs, including the heart, brain, lung, liver, kidney, spleen, thymus, and aorta. Total RNA from each organ was amplified as indicated before for microdissected samples. Finally, the Pig Universal Reference was made by equimolar mix of aRNA (first round and second round) of 8 swine control organs.

2) Preparation of the Labeled Pig cDNA Sample for DNA Chips Analysis

Fluorescently-labeled cDNA was prepared and purified according to an Agilent protocol (Agilent Direct-Label cDNA Synthesis Kit Protocol, Agilent, Palo Alto Calif.). 4 μg of swine aRNA and 2.5 μg of random hexamer (In vitrogen) were used per reverse transcription reaction. Cy3- and Cy-5 dCTP (NEN Perkin Elmer) was incorporated into cDNA during reverse transcription. For purification with QIAquick PCR Purification Kit (Qiagen), three washes with buffer PE were performed. Paired cDNA were dryed under vacuum in a rotary dessicator.

3) Preparation of the Hybridization Mixture and Hybridization

Agilent Human cDNA Microarrays (Agilent, Palo Alto, Calif.) were hybridized according to Agilent supplier instructions with minor modifications. Cyanine 3-/cyanine 5 labeled cDNA sample was resuspended in 5.96 μl of nuclease-free water and the following mix was added per sample

1.26 μl Deposition Control Targets (sp300 operon, Qiagen)

2.28 Cot-1 DNA (InVitrogen)

9.5 μl 2× Deposition Hybridization Buffer

After incubation at 98° C. for two minutes to denature the cDNA, and centrifugation at 10,000 g (13000 rpm) for 5 minutes, 16 μl on 19 μl of hybridization mixture were transferred in a new amber tube to eliminate the pellet. Finally 12 μl were applied to the microarray under a 24/30 mm coverslip (Corning) for 17 h at 60° C. in a waterbath, in Scienion hybridization chamber.

Each pig labelled sample was combined with the labelled Pig Universal Reference and the dye swap combined cy3/cy5 samples were hybridized on the two arrays on the same slide.

Washes were performed as recommended by Agilent supplier except that wash 1 was performed during 30 minutes twice and wash 2 was performed during 12 minutes twice. Finally slides were dried by centrifugation 10 minutes at 400 g at room temperature.

Slides were scanned with an Agilent scanner (Agilent G2565AA Microarray Scanner System), with a resolution of 5 microns. Signal extraction was performed with Feature Extraction version 5 (Agilent G2566AA Feature Extraction Software; Agilent Palo Alto, Calif.). Output files were XML and Text file and visual results. Configuration parameters were the following:

In the general configuration, “Spot finder”, “PolyOutlierFlagger” and “CookieCutter” were selected.

In Find Spots configuration, “Autofind corners” was selected with a “Dev Limit” of 70 microns.

In CookieCutter configuration, “Reject based on IQR” of 1.42 for Feature and Background was selected.

In the PolyOutlierFlagger configuration, “Non-Uniformity Outlier Flagging” and “Population Outlier Flagging” were selected with the default parameters.

4) Gene Expression Analysis

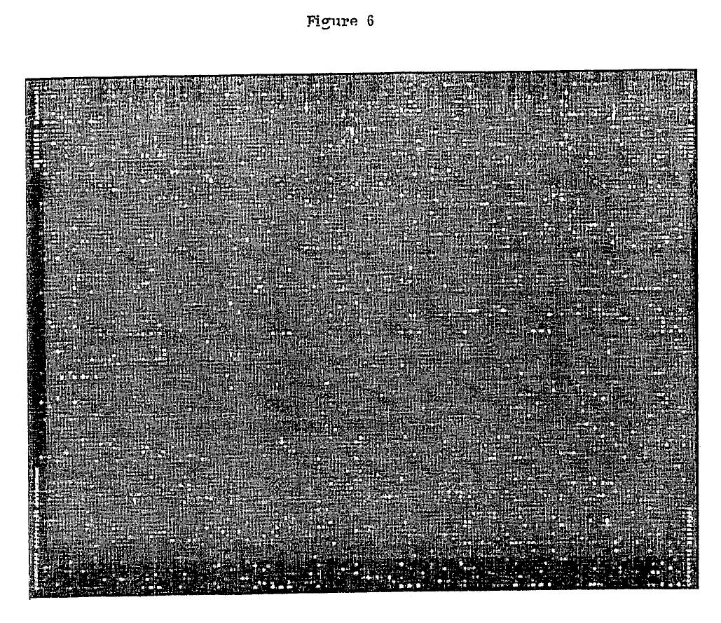

The gene expression patterns were individually determined for each sample. In a typical experiment, samples of three control pigs and four diet supplemented pigs were analysed in reference to the universal pig signature after 6, 9, 12, and 24 weeks of diet. Control samples were either circulating non activated monocytes from non diet pigs or laser captured endothelial cells from non diet pigs. FIG. 6 illustrates a typical expression signature, and table 2 hereunder indicates the set of genes that were reproducibly up regulated in an atherosclerotic plaque.

These genes which are named positive genes, were co-expressed with a set of canonical genes which are listed in table 2 hereunder. Table 2 hereunder indicates a combination of canonical genes and novel genes that were not described before to be up regulated during the progression of an atherosclerotic plaque and are co-expressed with reference genes that were known to be up regulated in an arterial plaque. This set of genes represents a novel gene signature for atherosclerosis and identifies different metabolic pathways that are positively regulated during the pathogenic process and contributes to the drastic changes in expression at the level of a vascular lesion where an atherosclerotic plaque can develop.

TABLE 2

| | Fold Ex- | | Fold Ex- |
|---|---|---|---|
| | pression | Novel Genes that are | pression |
| Reference | Mean | differentially expressed | Mean |
| Genes | value | In an Atherosclerotic plaque | value |
| ABCA1 | 3.0 | New genes | |
| CD68 | 2.1 | Stearoyl CoA Desaturase | 6.2 |
| CD36 | 4.7 | Phosphatidic acid phosphatase | 7.5 |
| | | 2B | |
| LDL - R | 2.0 | Phosphoinositide-specific- | 2.1 |
| | | Phospholipase C | |
| PECAM | 2.0 | New canonical genes | |
| HSP60 | 2.3 | Aldehyde dehydrogenase | 1.9 |
| HSP70 | 2.4 | Aldehyde Reductase AKR1 A1 | 2.0 |
| TLR4 | 1.9 | Aldose Reductase AKRI B1 | 2.0 |
| Erg 2 | 2.6 | Thymosine beta 4 | 5 |
| VEGF | 1.9 | Sphingomyelinase | 2.3 |
| Paraoxanase | 1.4 | Sphyngosine Phosphate liase | 3 |
| 3 | | | |
| BRCA1 | 4.2 | Acide Ceramidase | 2.1 |
| HIF1 | 2.3 | UDP-glucose ceramide glucosyl | 2.2 |
| | | tranferase | |
| IL1 - R | 1.5 | ATPase Ca++ transport | 6.5 |
| | | binding protein 1 | |
| ATF3 | 1.6 | CD 163 | 3.05 |
| NADH | 2.1 | | |
| dehydrogena [symbol] e | | | |
| HCTGF | 2.4 | | |
| Galectine 3 | 3.0 | | |

### Example 3

### Novel Genes Associated with the Progression of an Atherosclerotic Plaque

A—PCA Analysis of Gene Expression Data

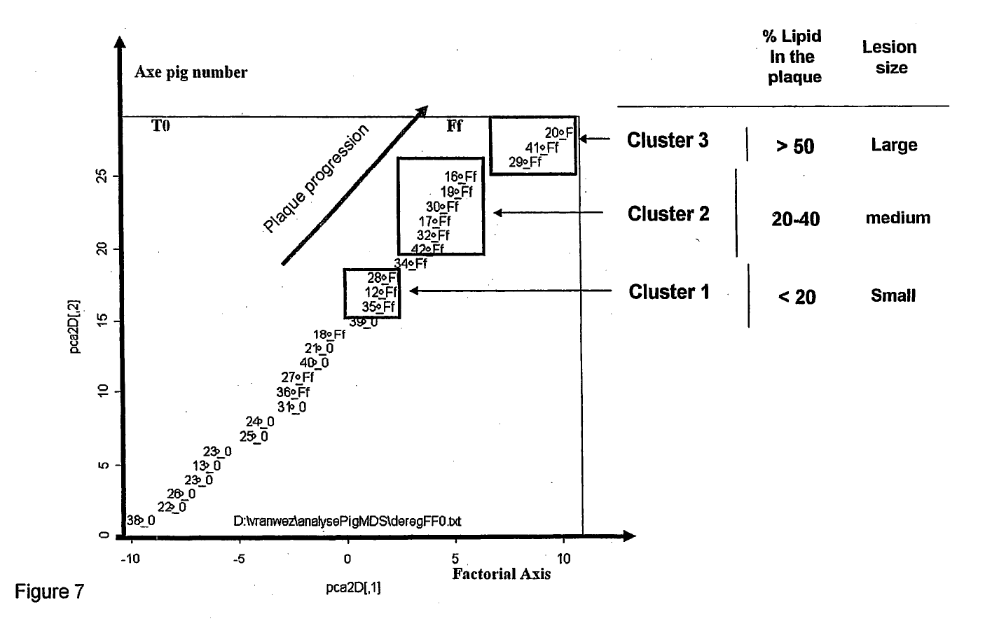

FIG. 7 illustrates a factorial analysis of 24 different pigs with 1200 genes that were stastically up and down regulated during the progression of an arterial plaque. Stastical analysis was performed to evaluate lacking values with k nearest neighbours method (k=10) followed by a bilateral student test (1%) with Welch approximation (without ratio threshold). This method allowed the identification of significantly deregulated genes between the different pigs. The PCA method was used to transform a 24×1200 matrix into a 24×1 matrix. This analysis clearly shows that the diet minipigs can be clustered into three separate groups. Group 1, 2 and three can also be associated with different phenotypic attributes such as the amount of lipid present in the plaque, and the size of the plaque. Gene expression analysis of these different groups clearly establishes the association of an increased expression of Stearoyl CoA desaturase, phosphatidic acid phosphatase and Phosphoinositide specific phospholipaseC-B1 with the progression of the plaque (table 3).

Table 3 hereunder shows fold increase of gene expression of Stearoyl CoA desaturase, phosphatidic acid phosphatase and Phosphoinositide specific phospholipaseC-B1 in macrophage rich vascular lesions relative to their expression in control monocytes.

TABLE 3

| Pig cluster | | Phosphatidic | Phosphoinositid |
|---|---|---|---|
| according to | Stearoyl coA | acid | e-specific- |
| | deasturase | phosphatase | phospholipase C |
| Group I | 6.2 | 7.5 | 2.6 |
| Group II | 13.6 | 7.2 | 2.4 |
| GroupIII | 17.5 | 8.7 | 2.8 |

B—New Series of Genes

1) Stearoyl CoA Desaturase

Stearoyl CoA desaturase is differentially expressed at the cellular level in early lesions, containing activated endothelial cells and macrophages, together with genes that are known to be involved in the process of atherosclerosis,

The exact mechanism by which stearoyl CoA desaturase may influence plaque growth and instability is unknown.

Stearoyl CoA deasaturase is a member of a family of genes that are regulated by sterol regulatory element-binding proteins (SREBPs). This includes, acetyl CoA carboxylase (ACC), fatty acid synthase (FAS), glycerol 3-phosphate acetyltransferase (GPAT) and Delta 6 and Delta 5 desaturases.

Stearoyl CoA desaturase is the rate limiting enzyme in the biosynthesis of monosaturated fatty acids. It catalyzes the formation of palmitoleate (delta 9, 16:1) and oleate (delta 9, 18:1) from palmitate (16:0) and stearate (18:0) which are the major constituent of membrane phospholipids and triacylglycerol stores found in adipocytes (Kasturi R and Joshi V. C., 1982, JBC, 257, 12224-12230 ; Ntambi J. M., 1995, Prog. Lipid Res., 34, 139-150).

Stearoyl CoA desaturase has been shown to play a role in lipogenesis and in adipocyte differentiation. Gene expression is elevated in liver tissue and adipose tissue and has been shown to control the serum level of triglycerides and fatty acids (Jones B. H. et al 1996, Am J. Physiol., 272, E44-E49; Pan D. A. et al 1994 , J. Nutr. 124, 1555-1565 ).

The role of Stearoyl CoA desaturase in hepatocyte triacylglycerol metabolism and in adipocyte differentiation is well documented. Transcriptional up regulation is induced by dietary factors, metals, peroxisomal proliferators, hormone such as insulin and metabolites such glucose (Park E. I., et al 1997, J. Nutr. 127, 566-573;/Casimir D. A., & Ntambi, J. M., 1996, J.B.C., 271, 29847-29853;/Ntambi J. M. et al 1996, Biochem. Biophys. Res. Com. 220, 990-995). Down regulation is observed in the presence of polyunsaturated fatty acids and during adipose tissue differentiation. Thus, Stearoyl CoA desaturase exhibits target characteristic for the treatment of obesity. Targeted disruption of the gene in a mouse model revealed that the enzyme plays a direct role in the biosynthesis of cholesterol ester, triglyceride and wax ester. Stearoyl CoA desaturase deficient animals, are deficient in hepatic cholesterol and triglycerides. The mice are leaner than normal and exhibit defects in lipid metabolism (Miyazaki M et al, 2001, J. Nutr. 131, 2260-2268 ; Ntambi, J. M. et al 2002, PNAS, 99, 11482-11486).

The present invention is based on the unexpected discovery that beside the hepatic and the adipocyte tissues, Stearoyl CoA deasturase is differentially expressed in tissues that are constitutive of an early atherosclerotic plaque, in hyperlipidemic conditions that are relevant with the development of atherosclerosis, and is co-expressed with known, phosphatidic acid phosphatase and Phosphoinositide specific phospholipaseC-B1. Together, the presence of these three enzyme identifies the synthesise of diacylglycerol as a key step in the accumiulation of lipid vesicles. These proteins are also assocoiated with atherosclerosis-associated genes in the same injured tissue. This allows the identification of a target pathways that is useful for the identification of agents with both diagnostic and therapeutic activity in atherosclerosis.

Mechanisms controlling transcriptional up regulation of the Stearoyl CoA desaturase gene during the growth of an atherosclerotic plaque are unknown. Using permanent cell lines, it was shown that expression of Stearoyl CoA desaturase is negatively regulated by PPAR gamma agonists such as thiazolidinediones during adipocyte differentiation (Kim Y-C et al , 2000, J. Lipid Res. 41, 1310-1316 ). This contrasts with known effects of PPAR gamma agonists on atherosclerosis associated genes expression during macrophage differentiation and foam cells formation in an arterial plaque. Thus Stearoyl CoA desaturase may have different effects in liver cells, vascular cells and adipocytes and may exert a specific role in the development and the stability of an atherosclerotic plaque.

Based on this unexpected up regulation in the atherosclerotic lesion, the present invention provides methods to monitor the differential expression of Stearoyl CoA desaturase for diagnostic and prognostic purpose and to identify compounds that are capable to increase or decrease the activity of Stearoyl CoA desaturase in association with phosphatidic acid phosphatase and Phosphoinositide specific phospholipaseC-Bi to specifically reduce the size of a plaque, its erosion and to stabilize the plaque.

2) Phosphatidic Acid Phosphatase and Phosphoinositide Specific Phospholipase C

Both enzymes are implicated in the production of diacylglycerol (DAG). Phosphatidic acid phosphatase is involved in the synthesis of DAG from the lysophosphatidic acid and the phosphatidic acid. Phosphoinositide specific phospholipase C is involved in the production of DAG by the specific hydrolysis of phosphatidyl inositol. Together with the high expression of stearoyl coA desaturase, this clearly establishes for the first time, the observation that a large quantity of stearoyl-diacylglycerol is produced in the macrophage foam cell. This identifies a new target pathway for the identification of products that are able to prevent this accumulation of diacylglycerol and the concomitant formation of lipid vesicles in foam cells.

C—New Canonical Genes Associated with Plaque Progression

1) Aldehydes Reductase: Aldo Keto Reductase Family 1 Member B1: AKR1B1 (EC 1.1.1.21); Aldo Keto Reductase Family 1 Member A1: AKR1A1 (EC 1.1.1.2)

AKR1A1 and AKR1B1 are members of the aldo ketose reductase super family which includes a number of related monomeric NADPH-dependent oxidoreductases such as aldose reductase, xylose reductase, prostaglandin F reductase, and many others (Jez J. M. et al 1997 Biochemical Pharmacology 54, 639-647). The enzymes are closely related monomeric proteins but exhibit different substrate specificity. AKR1B1 is a low Km aldose reductase enzyme and is involved in the polyol pathway. The enzyme controls the reduction of aldose such as glucose and galactose to their corresponding polyol such as sorbitol and galactilol.

This enzyme controls the level of glucose in the blood and exhibits the characteristics of a pharmacological target for treating diabetes and its hyperglycaemic complications such as, neuropathy, retinopathy, nephropathy, and micro angiopathy (Mylari B, J, US 20020143017).

Its precise function in the pathogenesis of atherosclerosis and more specifically, in the progression of a plaque is totally unknown.

AKR1A1 is a high Km aldose reductase. At elevated blood glucose levels, a significant flux of glucose through the polyol pathway is induced in tissues like nerves, retina, lens and kidney. Activation of the polyol pathway is therefore considered to induce diabetic complications. Aldose reductase inhibitors are used to prevent or reduce these complications. These inhibitors however, demonstrate an imperfect control of blood glucose and their beneficial effects are far satisfactory. Two main classes of orally active aldose reductase inhibitors have been reported, with Sorbinil and Tolrestat being the most representative members of each family. The in vivo activities of these two products are very different and some of them have been shown to cause liver complications and hypersensitivity reactions when used to control glucose production in diabetes patients (Costantino L, et al 1997, Exp. Opin. Ther. Pat. 7, 843-851). For these reasons, the search for new molecules with better pharmacological properties against these reductase is an active area.

In addition to their implication in the polyol pathways, AKR1 A1 and AKR1 B1, express an aldehyde reductase activity with different substrate specificity. Both enzyme are implicated in the glycerolipid pathway and catalyze the reduction of lipid derived aldehyde to generate glycerol. Glycerol is involved in the biosynthesis of diacyl glycerol and triacylglycerol via the production of glycerol 3 phosphate and the metabolism of glycerolipids. Therefore, activation of the AKR1 A1 and/ or AKR1 B1 aldehyde reductase activity in macrophages during the development of the atherosclerotic plaque may be responsible for an over expression of diacyl glycerol and the accumulation of foam cells at the level of the plaque.

Oxidation of circulating Low Density Lipoprotein (LDL) and their uptake by macrophages via scavenger receptors is the major reaction that promotes the recruitment and accumulation of lipid-laden macrophages in the vessel wall, leading to fatty streaks that precede the development of a plaque. Lipid peroxidation which occurs in these foam cells during atherosclerosis generates high concentration of breakdown products which may be toxic or mitogenic to other vascular cells and may be responsible for the progression of the plaque. Among these down products, aldhedydes are the end products of lipid peroxidation and exhibits high reactivity with different biomolecules that may be implicated in the pathogenesis of atherosclerosis. Unsaturated aldehydes for instances, are derived from the oxidation of poly unsaturated fatty acids such as linolenic and linoleic acids which are particularly abundant in oxidized LDL (Morisaki N. et al, 1985, J. Lip. Res. 26, 930-939).

Therefore, in addition to being responsible for the production and accumulation of high amount of active glycerol and di and triacyl glyceryl in foam cells, activation of the aldehyde reductase activity may generate intracellular or secreted active down products that may activate the atherosclerotic process. The exact mechanisms by which these aldehydes regulate the growth, the stability or the regression of an atherosclerotic plaque are totally unknown. Reactions other than the polyol pathway may be activated by the AKR1 family.

AKR1 A1 and AKR1 B1 have different substrate specificity. In addition, major differences exist in the function and tissue specific expression of aldehyde reductase and aldose reductase (O'Connor T et al, 1999, Biochem J. 343, 487-504). It was recently shown for instance, that perfusion induced ischemia influences aldose keto reductase but not aldehyde reductase activity in heart. Specific aldose keto reductase inhibitors were cardioprotective. The activation of aldose reductase activity in ischemic heart was not due to increased expression but to activation of the enzyme by endogenous factors (Hwang, Y. C., December 2001, FASEB J., 10. 1096). Thus aldose reductase and aldehyde reductase activities clearly express different tissue specific functions and are clearly involved in different pathways. AKR1 A1 preferentially catalyzes the NADH dependent reduction of aliphatic aldehydes, aromatic aldehydes, and biogenic amines. While AKR1 B1 expresses also an aldehyde reductase activity, the enzyme better catalyzes the NADH dependent reduction of aldopentoses, aldohexoses. Therefore, while both enzymes catalyses the reduction of lipid derived aldehydes, AKR1 A1 appears to be a better enzyme for aldehyde substrates. Using molecular docking and data base screening, it was recently shown that new series of inhibitors with a better specificity to the aldose reductase AKR1 B1 when compared to aldehyde reductase activity of AKR1 A1 could be designed, suggesting that the reverse strategy might be possible (Rastelli G. et al 2002, Bioorganic & Medicinal Chemistry 10, 1437-1450)

The present invention demonstrates for the first time that these enzymes are up regulated at the transcription level in early and advanced atherosclerotic plaques under conditions of hypercholesterolemia and in the absence of high level of blood glucose and insulin. This suggest that these enzymes may have a specific implication in the macrophage dependent lipid metabolism.

Therefore, the present invention, relates to compounds and methods using the differential expression of an aldehyde or aldose reductase activities in an atherosclerotic plaque relative to their normal expression to discover new products that specifically reduce this reductase activity, to prevent or control the production of lipid dependent aldehyde derived down products.

Specifically, the present invention identifies ways to treat patients with atherosclerosis in the absence of increased levels of circulating triacyl glycerol and glucose thus allowing treatment of atherosclerosis in the absence of hyperglycemia and avoiding potential metabolic side effects of drugs that lower sorbitol and are normally used for the treatment of hyperglycemia. This invention relates to pharmaceutical compositions that contain a specific aldehyde reductase inhibitor and to methods using such compositions to treat or prevent the accumulation of foam cells, the progression and the instability of an atherosclerotic plaque in mammals under hypercholesterolemic conditions.

2) Aldehyde Dehydrogenase, ALDH1 (EC 1.2.1.3)

Aldehyde dehydrogenase is one of the major enzyme of the alcohol metabolism, next to the alcohol dehydrogenase (ADH 103700). The protein belongs to the NAD-dependent aldehyde dehydrogenase family which contains ALDH I, II, III, and IV encompassing over twenty different isoforms.

The catalytic role of ALDH is well known. ALDH is the enzyme that catalyzes the hydrolysis of esters as well as oxidize aldehydes into acids. The enzyme has been found to be involved in different metabolic pathways, including the fatty acid pathway, bile acid biosynthesis, glycerolipid metabolism, tryptophan metabolism, among others.

An inactive dominant mutant form of ALDH1 was described in 1979 in Asian populations (Goedde et al Hum Genet. 51, 331-334). Loss of enzymatic activity in these individuals was the result of a point mutation (Yoshida et al, 1984, Proc. Natl. Ac. Sci. USA 81, 258-261). Interestingly enough, this inactive mutant did not display any metabolic abnormalities. Therefore, this molecule appears to be an excellent target for the design of small molecules to control its activity in patients.

For a long time, this enzyme was considered as a target for the treatment of patients with alcohol sensitivity and for the treatment of alcoholism and alcohol abuse. The present invention describes for the first time, a positive differential expression of this enzyme in an atherosclerotis plaque.

The role of ALDH1 in the development of atherosclerosis is totally unknown.

ALDH1 is cytosolic, exhibit a high Km for acetaldehyde and has been assigned a major role in glyceraldehydes detoxification. The enzyme has two distinct catalytic activities and exhibit both esterase and dehydrogenase activities (Duncan R J; 1983 Biochem .J. 230, 261-267 and Tu G C and Weiner H. 1988, J. Biol. Chem., 263, 1218-1222). The existence of specific inhibitors of the esterase and the dehydrogenase activities has been demonstrated (Abriola and Pietruszko, 1992, J. Protein Chem., 11, 59-70). Accumulation of acetaldehyde in blood is observed when ethanol is ingested and is accompanied by marked increases in heart rate and cardiac output as well as by decreases of vascular resistance. These changes were reversed by inhibiting ALDH1 activity (Kupari et al 1983, Alcohol Clin Exp Res 7, 283-288).

Alternatively, ALDH1 is also involved in the fatty acid metabolism pathway and is reported to generate aldehyde derivatives from fatty acid. Thus the presence of ALDH1 in a growing atherosclerotic plaque, may be responsible for the production and accumulation of cytotoxic aldehyde derivatives.

3) Thymosin β 4

Thymosin β 4 is a member of the Thymosin super-family which comprises highly conserved polar polypeptides ranging in molecular weight from 1 to 15 kDa, and originally thought to be thymic hormones. In 1990, Thymosin β 4 was identified as an intracellular G actin sequestering peptide (Safer D., and Golla V. T., 1990, PNAS, 87, 2536-2540).

Thymosin β 4 has been reported to have an effect on the differentiation of T lymphocytes (Low, T. L. K. et al, 1981, PNAS, 78, 1162-1166), and to inhibit the migration of macrophages (Weller F. E., et al, 1988, J. Biol. Resp. Modif. 7, 91-96). More recently, Thymosin β 4 has been shown to stimulate endothelial cells attachment and spreading and to increase the production of matrix metalloproteinases that may degrade the basement membrane (Grant D. S. et al, 1995, J. Cell Sci. 108, 3685-3694 , Malinda K. M. et al 1997, FASEB J. 11, 474-481). It was finally shown that Thymosin β 4 sulfoxide can be produced by monocyte and act as an anti-inflammatory agent (Young J. O. et al, 1999, Nat. Med. 5, 1424-1427).

The exact mechanism by which Thymosin β 4 influences cell migration and spreading was established in 1991 (Safer D., et al J.B.C. 266, 4029-4032). The molecule forms a I:I complex with G-actin and inhibits G actin polymerization, a specificity shared with other members of the thymosin family. In vivo experiments with leucocytes, have indicated that Thymosin β 4 is in fact the main G actin sequestering molecule (Cassimeris L. 1992, J. Cell Biol. 119, 1261-1270). Over expression of the molecule in permanent cell lines, causes the cells to spread out more fully and to adhere more strongly. This observation suggested that Thymosin β 4 may also act as an anti apoptotic mediator (Niu, M., et al, 2000, Cell Adhes. Commun. 7, 311-320).

The role of Thymosin β 4 in the development of an atherosclerotic plaque is totally unknown. The functional implication of this molecule in the progression of the disease may be multiple.

Different possibilities, but not limited to, are described in the following:

First, it has recently been shown that agents that disrupt the actin cytoskeleton organization including cytochalasin B, myosin light chain phosphatase, myosin light chain kinase inhibitors and simvastatin, up regulate endothelial cell Nitric Oxide Synthase (eNOS) (Liao J. K. U.S. Pat. No. 6;423,751). It is well established that eNOS activity is a major component of the atherogenic process (O'Driscoll G. et al, Circulation, 95, 1126-1131). Endothelial cells derived NO inhibits pro-atherogenic components including oxidative modification of LDL and adhesion of monocytes (Cox D. A. and Cohen M. L., 1996, Pharm. Rev., 48, 3-19; Tsa P. S. et al, 1994, Circulation, 89, 2176-2182).

Therefore, as a regulator of G actin polymerization, Thymosin β 4 may be involved in the up regulation of eNOS and may function either as an anti or a pro atherosclerotic molecule.

Second, survival and cell death machineries are both induced upon stimulation of endothelial cells with oxidized LDL and other stress agents. In vitro and in vivo studies in animal models or cell culture have indeed shown that endothelial cells apoptosis is initiated at sites that are prone to atherosclerosis and further development of atherosclerotic lesions, correlates with apoptosis and cell death (Isner, M et al , 1995, Circulation, 91 , 270-2711; Claise C, et al, 1999, Atherosclerosis, 147, 95-104; Dimmeler J, et al, 1997, Circulation, 95, 1760-1763). Down stream effectors of apoptosis, such as p38 MAP kinase, p53 and capsases are induced upon exposure to oxLDL and stress factors (Jing Q et al , 1999, Circ. Res., 84, 831-839; Napoli C et al. 2000, Faseb J., 14, 1996-2007; Xiuwu Zhang M D et al, 2001, Circulation, 104, 2762-2771). Concomitantly, oxLDL can stimulate the expression of the Zn finger transcriptional factor ATF3 and the Integrin Linked Kinase (Nawa T et al, Atherosclerosis, 2002, 161, 281-291 ; Kawauchi J et al, JBC 2002 In press). Both proteins are expressed in atherosclerotic lesions, correlate with the presence of dead cells and have been shown to regulate p38, p53 and capsase apoptotic activities. Therefore, initiation of atherosclerosis may be the result of a conflicting unbalance between apoptosis and survival, leading to vascular injury. Suppression of p38 activity and other effectors of the apoptotic machinery may constitute a feed back mechanism to protect the endothelium against oxLDL induced injury. Delineating the mechanisms that control the balance between survival and apoptosis may therefore be a fruitful approach for the discovery a new therapeutic windows and new products. Endothelial cells survival is maintained by contact to extra cellular matrix. In the absence of adhesion, endothelial cells rapidly undergo apoptosis, a phenomenon called anoikis. Integrin mediated signals are required to maintain endothelial cells integrity and reduce the sensitivity to stress. Adhesion involves focal plaque formation, activation of ILK and is probably essential in maintaining an anti atherogenic status. G actin polymerization-depolymerization is a major reaction that control cell spreading and proliferation. Therefore, Thymosin β 4 may stimulate a more complete and stronger spreading and adhesion of endothelial cells at sites of vascular lesions. Thymosin β 4 may thus act as a survival effector and prevent endothelial cells from apoptosis and cell death.

Third, It was shown that growth factor such as the Hepatocyte Growth Factor (HGF) can up regulate the expression of Thymosin β 4 in human umbilical vein endothelial cells (Oh, I, et al Biochem., Biophys., Res. Commun. 2002, 16, 296 (2):401). HGF can stimulate the invasiveness of monocytes at sites of atherosclerosis and was shown to expressed in atherosclerotic plaques (Beilmann M. 2000, Blood, 95, 3664-3669). Thymosin β 4 may thus be involved in monocyte macrophage and lymphocyte adhesion and migration at site of atherosclerosis thus contributing to plaque growth and instability.

In summary, the present invention describes for the first time a differential expression of Thymosin β 4 in an early and advanced atherosclerotic lesion. Thymosin β 4 may be considered for its development as an anti atherosclerotic target. The invention therefore includes methods and composition for the treatment of atherosclerosis and its clinical complications by controlling Thymosin β 4 activities. The invention includes the control of the progression, erosion, and regression of an atherosclerotic plaque.

4) Sphingomyelinase (EC 3.1.4.12), Acide Ceramidase (EC 3.5.1.23), Sphingosine Lhosphate Liase (EC 4.1.2.27) UDP-Glucose Ceramide Glucosyl Tranferase (EC 2.4.1.80)

Sphingomyelinase, acide ceramidase, UDP-gluclose ceramide glycosyl transferase and sphingosine phasphate liase are all important enzymes of the ceramide and sphingolipids metabolisms. The present invention indicates that these enzymes are up regulated at the level of transcription during the progression of an atherosclerotic plaque. The role the ceramide and sphingolipids patways in the process of atherosclerosis is totally unknown.

The enzyme sphingomyelinase catalyzes the hydrolysis of sphingomyelin to ceramide and choline phosphate. Different sphingomyelinase have been identified which can be separated into mitochondrial, lysosomal, cytosolic and secreted enzymes. Different and opposite functions have been ascribed to sphyngomyelinase. A role in cholesterol transfert from lysosome to the membrane has been found (Leventhal et al 2001, J. Biol. Chem. 276, 44976-44983). Uptake of oxidized LDL inhibits Lysosomal sphingomyelinase and causes accumulation of unesterified cholesterol in permanent cell line (Maor et al, 1995, ATVB, 15, 1378-1387). On the other hand, extracellular sphingomyelinase converts lipoproteins into potent atherogenic aggregated LDL (Marathe et al, 2000, ATVB, 20, 2607-2613).

Alternatively, the production and accumulation of ceramide and sphingolipid derivatives may have different consequences during the progression of a plaque. Ceramide is an important messenger of apoptosis and cell proliferation (Mathias S. et al, 1988 Biochem J. 335, 465-480) and elevated levels of ceramide in post-mortem samples of plaques in patients who died of atherosclerosis have been reported (Schissel S L et al, 1996, J. Clin. Invest. 98, 1455-1464).

Thus activation of the ceramide pathways at the transcriptional level may have a direct consequence on the accumulation of macrophages and foam cells at sites of atherosclerosis. Therefore, ceramide accumulation may constitute a high risk factor for plaque instability and erosion.

Alternatively, ceramide glycosil transferase catalyzes the formation of glucosylceramide An excess production of glucosylceramide may then be responsible for an excessive accumulation of second messengers like gangliosides or globosides.

Thus the present invention identifies the sphingomyelinase/ceramide/ceramide glucosyl transferase as a potential target pathway for controlling the progression of an atherosclerotic plaque.

5) CD163

CD163 is an inducible member of the scavenger receptor family (Law S K et al 1993, Eur. J. Immunol. 23, 2320-2325). This receptor is induced in CD14 positive macrophages by glucocorticoids and interleukin 10. and this induction is at least in part due to increased levels of RNA and protein (patent, WO 20010041177).

The potential role of CD163 in the process of atherosclerosis is totally unknown.

### Example 4

### Gene Expression in a Foam Cell Model

A series of primary or permanent cell lines can be used to monitor the formation of lipid vesicles in association with the differential expression of genes that are directly involved in the progression of an atherosclerotic plaque. All cells should have the capacity to incorporate fatty acids, Lipoprotein, modified lipoprotein including oxydized acetylated lipoproteins, Triglycerides, chilomicron and to exhibit vesicules that are characteristic of an atherosclerotic plaque associated foam cell. This includes but not limited to, HEP G2, U937, KG1, and THP1, HUVEC, Smooth muscle cells and adipocyte cell lines. In the present example, THP1 cells were used as a paradigm to generate an expression system which can mimic the formation of a foam cell in the plaque and can be used for a large scale screening of molecules that can inhibit or control the formation of a foam cell, via the control of vesicle accumulation and the expression of at least two of the proteins encoded by novel and canonical genes.

1) Cell Culture

The THP-1 cell line from the European Collection of Cell Cultures, (ECACC, Wilshire, UK) were selected to generate a cellular model that mimics the differentiation and the growth of a foam cell. Typically, two different culture conditions can illustrate the production of these cells. The cells (5.10⁵ cells/ml) can be maintained and grown in RPMI-1640, 10% FBS, 100 Unit/ml penicillin and 100 μg/ml streptomycin, 200 mM L-Glutamine (Biowhittaker, Verviers, Belgium) in 37° C., 5% CO2 incubator. The medium can be supplemented by either oxidized lipoproteins or specific fatty acids. Medium was replaced every 2-3 days.

2) Isolation and Modification of Lipoproteins

Human LDL were isolated from fresh plasma using a two steps KBr gradient ultracentrifugation (Leger et al Free Rad Res. 2002, 36, 127-142 ) LDL were dialyzed against NaCl 150 mM, sodium phosphate 10 mM, DTPA 10 μM (pH 7,4) for 24 hours. Copper oxidized LDL was prepared under sterile conditions by incubating 0.2 mg/ml of LDL with 5 μM CuSO4 for 16 hours at 37° C. At the end of this incubation, oxidation was stopped by addition of BHT (40 μM final) and DTPA (100 μM final). OxLDL were extensively dialysed against NaCl 150 mM and Sodium Phosphate 10 mM (pH 7,4) for 24 hours. All preparations were filtered through 0.4 μm filters.

LDL and oxLDL were extensively characterized by measuring the concentration of ApoB, total proteins, total cholesterol and vitamin E, the apparition of conjugated dienes (D0 at 234 nm) and the determination of fatty acid and oxysterol composition (see table 4).

TABLE 4

| | Protocols | LDL | oxLDL |
|---|---|---|---|
| ApoB | Immuno nephelometry | X | |
| Protein | Lowry-Maxwell | X | |
| Cholesterol | Enzymatic protocol | X | |
| Triglycerides | Chromatography | X | X |
| Fatty acids | HPLC | X | X |
| Vitamine E | Under validation | X | X |
| Oxysterols | Agarose gels | X | X |
| Electrophoretic | Spectrophotometry | X | X |
| Spectra 200-400 nm | OD | | X |
| Dienes conjugates | | | |

Finally, lipoproteins were also characterized by their electrophoretic mobility.

Labelling of OxLDL with Cyanine 3 succinimidyl ester (Amersham Pharmacia Biotech) was prepared as described (Stanton et al. JBC, 11992, 267, 22446-22451). At the end of the labelling procedure, Cy3-OxLDL were extensively dialysed and labelling efficiency was evaluated by measuring the absorbance at 548 nm.

3) Preparation of Fatty Acids

Fatty acids can be prepared according to Spector A A and Hoak J C, (1969 Anal. Biochem, 32: 297-302): Breifly, 100 μmol of fatty acid (12:0, 16:0, 18:0 and 20:0) can be dissolved in 7.5 ml of hexane containing 400 mg of Celite (Sigma). The solvent is then evaporated under nitrogen by continuous magnetic stirring. The fatty acid-coated particles are then mixed with fatty acid free albumin, in serum free medium for 1 hour at room temperature under nitrogen. After centrifugation, the supernatnts containing fatty acid coupled to albumin is conserved.

4) Foam Cell Formation in the Presence of Fatty Acid or oxLDL and RNA Extraction

Vesicle formation in the presence of fatty acids was induced as follows: briefly, THP1 differentiation was induced in a medium supplemented with 10⁻⁷ M of phorbol 12-myristate-13-acetate (Sigma) for 24 hours at 37° C., 5% CO₂. Differentiated-THP-1 was incubated with or without 200 μM of fatty acid-BSA complexes for 24 hours at 37° C., 5% CO₂. After washing and paraformaldhehyde fixation, cells were stained with Nile Red (1 μg/ml) and Hoechst 33342 (10 μg/ml) solution for 10 minutes at RT. After washing, images of cells staining with Nile Red and Hoechst 33342 were automatized captured using a fluorescence microscope controlled by MetaMorph Software (Universal Imaging) and coupled with a CCD camera. After images analysis, results were expressed as the sum of Nile red intensity per cells number.

To induce differentiation and foam cell formation in the presence of oxidized LDL the following procedure was used: Breifly, 2.10⁶ cells/wells were plated in 6-well plates in RPMI 1640, 5% FBS supplemented with 10⁻⁷ M of phorbol 12-myristate-13-acetate (Sigma) for 24 hours at 37° C., 5% CO2. Cells were washed with 1 ml of pre-warmed medium and maintained in 2 ml of pre-warmed medium for 24 hours at 37° C., 5% CO2 to reduce specific phorbol 12-myristate-13-acetate activation. Differentiated THP-1 were incubated with low density lipoproteins (native LDL and oxLDL at 10 μg/ml and 100 μg/ml) or lipoproteins buffer in RPMI 1640, 5% FBS medium for 6 hours At the end of each stimulation point, cells were washed once with 2 ml of PBS, pH 7,4 and lysated by Trizol. RNA extractions were performed according the instructions of manufacturer.

Quality controls were performed in parallel on foam cell formation and cell viability. Briefly, cells were fixed with paraformaldhehyde 2% for 15 minutes at RT, washed twice with H2O and stained with Oil Red O solution to visualize intracellular lipids. Cells were counterstained with Mayer's hematoxylin for 10 minutes at RT following by fourth washing with H2O. Images of foam cell formation were captured using a microscope coupled with a color CCD camera and analysis software. Finally, for each stimulation point, the viability of cells was superior to 95% after Trypan blue exclusion.

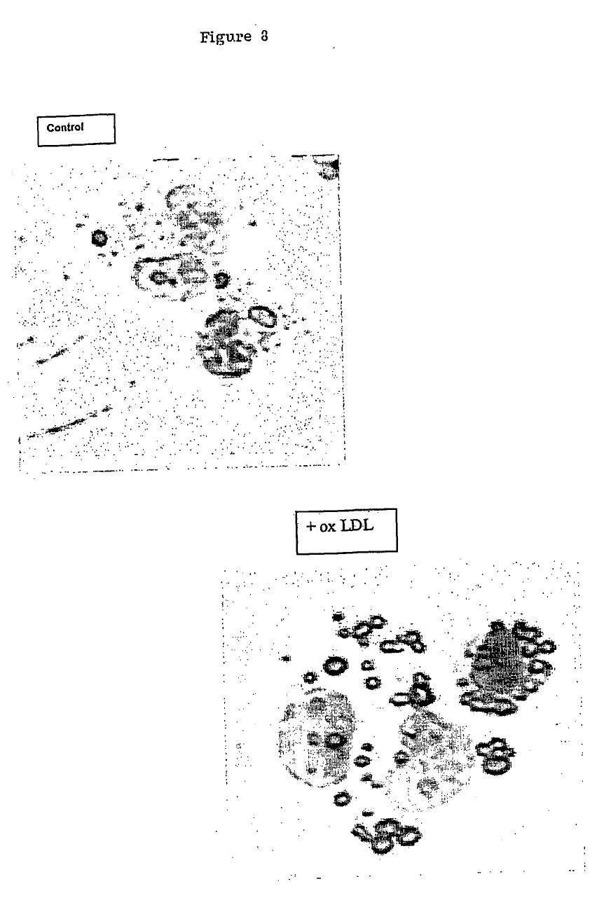

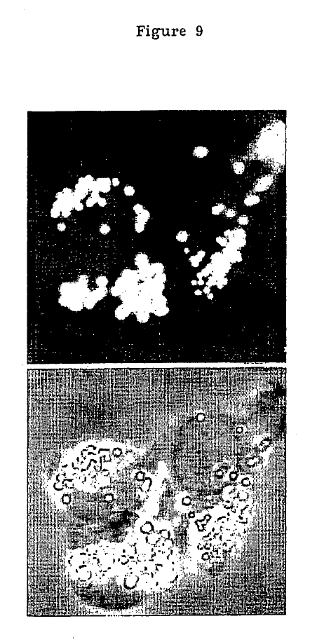

FIGS. 8 and 9 illustrate the uptake of oxLDL and fatty acid resulting in the formation of foam cells loaded with lipid vesicles. Formation of foam cells in FIG. 9 was obtained in the presence of stearic acid (18:0) a specific substrate of stearoyl coA desaturase.

5) Inhibition of Vesicle Accumulation

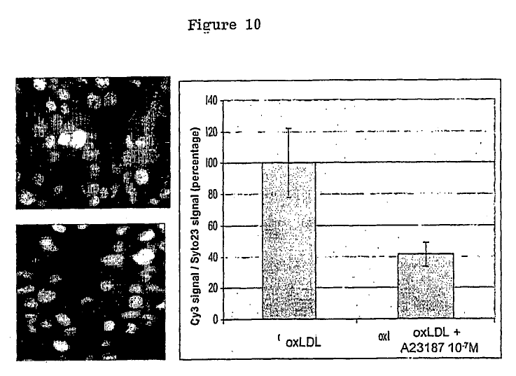

To induce differentiation, 8.5 10⁴ cells/wells were plated in 96-well plates in culture medium supplemented with 10⁻⁷ M of phorbol 12-myristate-13-acetate (Sigma) for 24 hours at 37° C., 5% CO2. Cells were washed with 200 μl of pre-warmed medium. PMA-differentiated THP-1 cells were incubated with Cy3 labelled oxLDL (30 μg/ml) in the presence or in the absence of specific inhibitor. In the example given in FIG. 10, A23187 was used as a test-compound time. Cells were washed twice with PBS and nucleus were counsterstained with 2.5 μM Syto 23 for 20 minutes, at RT. After washing twice in PBS, images of Cy-3 oxLDL uptake were captured using a fluoresecence microscope coupled with a CCD camera. Each image was analysed and quantified using QFluoro Software (Leica) similar results can be generated in the presence of stearic acid.

6) RNA and cDNA Preparation

10⁶ cells are extracted with 1 ml Trizol (Invitrogen) following manufacturer's instruction. RNA are resuspended in 20 μl RNase DNAse free H2O. cDNA where prepared as previously described (Chevillard et coll 1996) briefly 1 μg RNA was reverse-transcribed using random hexamers (PdN6 Roche Diagnostics) and 1/100 e of the CDNA was used in each PCR reaction (50 μl final volume). PCR was performed using SYBR Green PCR or Taqman Core reagent (Applied Biosystems France), on ABI PRISM 7000 sequence detector apparatus and analysed with the dedicaced software. PCR cycles consisted of an initial step of UNG amperase at 50° C. for 2 min and an initial denaturation step at 95° C. for 10 min followed by 40 cycles of denaturation at 95° C. for 10s and annealing-elongation at 60° C. for 1 min. MgCl2 concentrations were optimized for each primer set in order to minimize primer dimer formation and to reach the best amplification yield. For each amplification, the Ct value, representing the cycle at which a significant fluorescent signal is first detected, was measured. In a given sample, signals obtained for each gene were normalized to the signal obtained for a housekeeping gene (beta actin or GAPDH or beta 2 microglobulin) thus taking account of any variability in the initial concentration and quality of RNA. Finally, relative quantitation of gene expression was determined by reference to a calibration curve obtained from serial dilutions of RNA prepared from control samples expressing target gene at a high level and handled concomitantly with each RT-PCR reaction. Results were considered if the corresponding standard curve was perfectly linear, with an exponential growth of PCR products and if the level of expression of the sample was in the same range as those obtained for the standard curve.

RNA quality controls and concentration measurements were done with a bioanalyzer 2100 apparatus (Agilent, France). RNA ladder 6000 (Ambion UK) is used as a reference for quantification. Total RNA are analysed with the RNA nano labchip kit (Agilent France) For total RNA a ratio of 1 minimum between 28/18 S is considered as acceptable.

7) PCR Primer Design

PCR primers and taqman probes were designed with the help of primer express 2.0 software (Applied Biosystem). Primers were chosen spaning exons junction when the genomic sequence was known. The specificity of primers was checked after alignement with FASTA software in Genbank and after amplification PCR products were checked on a 2% agarose gel electrophoresis.

Chevillard, S.; Pouillart, P.; Beldjord, C., Asselain, B., Beuzeboc, P. ; Magdalenat, H.; Vielh, P. (1996) Sequential assesment of multidrug resistance phenotype and measurement of S-phase fraction as preditictive markers of breast cancer response to neoadjuvant chemotherapy (Cancer, 77, 292-300).

1) A method for identifying therapeutic agents for reducing and monitoring the growth, erosion, rupture or stability of an atherosclerotic plaque comprising the analysis of the differential expression of at least two genes coding proteins chosen among among Stearoyl CoA desaturase, Phosphatidic acid phosphate, and Phosphoinositide-specific-phospholipase-B1, eventually in association with the analysis of the differential expression of at least one gene coding a protein choosen in the group comprising Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163

2) The method of claim 1, wherein said analysis is carried out in human or animal cells, tissue sections or animal models.

3) A diagnostic method of artherosclerosis or cardiovascular disorders relating to the atherosclerotic plaque in a biological sample of a subject comprising the analysis of the differential expression of at least two gene coding a protein chosen among Stearoyl CoA desaturase, Phosphatidic acid phosphate, and Phosphoinositide-specific-phospholipase-B1, eventually in association with the analysis of the differential expression of at least one gene coding a protein choosen in the group comprising Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.

4) The method of claim 3, wherein said analysis is carried out in human cells or tissue sections.

5) The method of claim 1, wherein the analysis is performed at the mRNA or protein level.

6) The method of claim 1, which comprises:
providing a plurality of different ligands in the form of an array on a solid surface, said different ligands being complementary to different segments of at least two genes coding a protein chosen among Stearoyl CoA desaturase, Phosphatidic acid phosphate, and Phosphoinositide-specific-phospholipase-B1, and eventually to different segments of at least one gene coding a protein in the group comprising Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 or being complementary to different segments of at least one gene coding said proteins,
applying a sample solution potentially containing the targets of the ligands to the array of ligands under conditions which allow the interaction of said ligands and its target, and
measuring the interactions of the targets with the different ligands of the array

7) The method of claim 6, wherein the ligands are nucleic acid probes and the sample contains target nucleic acids in order to measure the hybridization of the probes with the target nucleic acids.

8) The method of claim 7, wherein the nucleic acid probes are oligonucleotides.

9) The method of claim 8, wherein the array comprises 2 to about 200 oligonucleotides localized in discrete location per square centimeter on the solid surface.

10) The method according to claim 6, wherein the sample is from a patient developing artherosclerotic plaque.

11) Method of screening compounds useful for the treatment of artherosclerosis or cardiovascular disorders relating to the atherosclerotic plaque comprising the analysis of the differential expression of at least two gene coding a protein chosen among Stearoyl CoA desaturase, Phosphatidic acid phosphate, and Phosphoinositide-specific-phospholipase-B1, eventually in association with the analysis of the differential expression of at least one gene coding a protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 in the presence of a test compound.

12) The method of claim 11, wherein said analysis is carried out in human or animal cells, tissue sections or animal models.

13) The method of claim 11, wherein the analysis is performed at the mRNA or protein level.

14) The method of claim 13, wherein the analysis is performed on a solid support.

15) The method of claim 11, which comprises :
providing a plurality of different ligands in the form of an array on a solid surface, said different ligands consisting of all or part of at least two gene coding a protein chosen among Stearoyl CoA desaturase, Phosphatidic acid phosphate, and Phosphoinositide-specific-phospholipase-B1, and eventually to all or part of at least one gene coding a protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163.
applying a solution containing a test compound to the array of ligands, and
measuring the interaction, such as the binding, of the test compound with the different ligands of the array.

16) The method according to claim 1, wherein the test compound is a protein or molecule of small molecular weight.

17) Method of screening compounds useful for the treatment of artherosclerosis or cardiovascular disorders relating to the atherosclerotic plaque according to claim 11 comprising:
providing an assay for at least two proteins chosen among Stearoyl CoA desaturase, Phosphatidic acid phosphate, and Phosphoinositide-specific-phospholipase-B1, eventually in association with at least one protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163
contacting said assay with a test compound, and
measuring the action of the test compound on the said protein in the assay.

18) The method of any of claim 1, wherein the analysis comprises the measure of the differential expression of at least two genes coding a protein chosen among Stearoyl CoA desaturase, Phosphatidic acid phosphate, and Phosphoinositide-specific-phospholipase-B1, eventually in association with the measure of the differential expression of at least one gene coding a protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 and the comparison of said measure with the normal expression of said protein in early and advanced atherosclerotic plaques containing macrophages, under hyperlypidemic conditions and in the absence of high levels of blood glucose and insulin.

19) The method of any of claim 1, wherein the analysis comprises the measure of the differential expression of at least two genes coding a protein chosen among Stearoyl CoA desaturase, Phosphatidic acid phosphate, and Phosphoinositide-specific-phospholipase-B1, eventually in association with the measure of the differential expression of at least one gene coding a protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 and the comparison of said measure with the expression of reference genes that are representative of an atherosclerotic plaque.

20) The method of claim 19, wherein said references gene include membrane associated genes such as CD68, CD36 which are both markers of the macrophage lineage; PECAM 1, a marker for endothelial cells; markers of the inflammatory response such as TLR4, HSP60 and HSP70, Galectin 3 and IL1-R; markers of the oxidative stress including HIF-1 and Paraoxanase 3, metabolic marker such as NADH dehydrogenase; lipoprotein receptors such as LDL-R and VLDL-R.

21) The use of a compound modulating the expression of at least two gene coding a protein chosen among Stearoyl CoA desaturase, Phosphatidic acid phosphate, and Phosphoinositide-specific-phospholipase-B1, and eventually modulating the expression of at least one gene coding a protein among Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 Stearoyl CoA deasturase, Aldose reductase and aldehyde reductase, Sphingomyelinase, Acid ceramidase, Ceramide glucosyl transferase, Sphingosin phosphate liase, Thymosine beta 4, Aldehyde dehydrogenase, ATPase Ca++ binding protein and CD163 or modulating the activity of said at least one protein for the preparation of a pharmaceutical composition useful for preventing and/or treating artherosclerosis or cardiovascular disorders relating to the atherosclerotic plaque.
